(19) United States
(12) Patent Application Publication
(10) Pub. No.: US 2013/0197557 A1
(43) Pub. Date: Aug. 1, 2013

(54) CONICAL TIP POLYMERIC LANCET

(71) Applicant: FACET TECHNOLOGIES, LLC, Kennesaw, GA (US)

(72) Inventor: Anthony P. CRUZ, Suwanee, GA (US)

(73) Assignee: FACET TECHNOLOGIES, LLC, Kennesaw, GA (US)

(21) Appl. No.: 13/752,793

(22) Filed: Jan. 29, 2013

Related U.S. Application Data
(60) Provisional application No. 61/592,178, filed on Jan. 30, 2012.

(51) Int. Cl. A61B 5/15

(52) U.S. Cl. CPC A61B 5/1411
USPC 606/181

(57) ABSTRACT

A lancet having a conical body incorporating multiple lancet gauge levels defining specific penetration depths to generate varying sample production. The conical body has a narrow tip and a large base, and may be formed of a polymeric material.

### CROSS-REFERENCE TO RELATED APPLICATION

This application claims the benefit of U.S. Provisional Patent Application Ser. No. 61/592,178 filed Jan. 30, 2012, the entirety of which is hereby incorporated herein by reference for all purposes.

### TECHNICAL FIELD

The present invention relates generally to the field of lancets, and more particularly to disposable lancet having a conical polymeric tip for piercing the skin to obtain a sample of blood as in blood glucose testing or blood typing analysis.

### BACKGROUND

Samples of blood must sometimes be collected from a human or animal subject. For example, many diabetics periodically monitor their blood glucose level by collecting a small blood sample from their fingertip, forearm, or other body part. In addition, small blood samples are also commonly collected prior to accepting blood from a donor at blood drives or blood banks, to determine the donor's blood type and/or to screen potential donors for anemia, diseases, or other conditions.

Generally, lancing devices are commonly used to pierce the skin of a subject to obtain a sampling of blood, interstitial fluid, and/or other bodily fluids. Typically, a lancing device incorporates a spring-driven carrier for holding a disposable lancet and some type of triggering mechanism to release energy stored in the spring to drive the sharp edge/point of the lancet to penetrate the subject's skin. Commercially-available lancets typically resemble the device depicted in FIGS. 1A and 1B. The depicted lancet 10 has a body 14 and a sharp tip 12 extending vertically from the top of the body. Commercially available lancets typically have sharp blade-edged or needle-pointed tips constructed of steel or other metal.

A desired effect of using lancets is to draw an amount of blood sufficient to perform medical tests. Different subjects or different testing sites on the same subject may generate differing sample sizes resulting from a lancing procedure that pricks the skin at a specified penetration depth and/or transverse dimension. For example, different test subjects may have differing capillary bed depths and/or differing blood flow responses from similar skin pricks. Similarly, a sample site on a subject's fingertip may generate a greater sample size, whereas a forearm or other alternate sample site may generate a lesser sample size, for the same subject. These differences in sampling response can necessitate a lancet penetration that is shallower/deeper or narrower/wider in some sampling applications than in others. For this reason, lancets with sharp tips of various thicknesses are available. Commercially available lancet thicknesses are commonly measured or specified by numerical gauge or wire thickness. Example gauge formats include 25, 28 and 33 gauge. The gauge scale is numerically arranged with larger numbers signifying thinner tips and smaller gauge numbers signifying thicker tips. For a given penetration depth, a larger gauge lancet will typically produce a smaller skin prick and lesser sample size, and a smaller gauge lancet will typically produce a larger skin prick and a greater sample size.

The provision of lancets of varying gauges for different sampling needs can lead to inefficiencies in use resulting from selection of an incorrect lancet gauge. For example, selection and use of too small a lancet may result in inadequate sample size, necessitating multiple lancing procedures. Alternatively, use of too large a lancet may result in too large a sample than needed and greater perception of pain by the subject, potentially reducing compliance with a prescribed testing regimen. The manufacturing and stocking of multiple lancet gauges may also increase costs and required shelf space.

Thus it can be seen that needs exist for improvements to lancets for blood sampling. It is to the provision of an improved lancet meeting these and other needs that the present invention is primarily directed.

### SUMMARY

In example embodiments, the present invention provides a conical polymeric lancet tip that incorporates multiple gauge numbers within a single body. The change in gauge number depends on the position along the conical body and is equivalent to the change of position depth.

In one aspect, the invention relates to a lancet with a body that has a tapered surface extending between a sharp tip and a wide base. The tapered surface is configured to define a plurality of different lancet gauge levels corresponding with a plurality of different penetration depths.

In another aspect, the invention relates to a lancet with a sharp tip that has a polymeric material. The sharp tip has an acutely-angled tapering outer surface and a base with a surface area that is larger than the sharp tip. The lancet also has a lancet body that is integrally formed with the sharp tip.

In a further aspect, the invention relates to a method of controlling penetration depth of a lancet. The method includes configuring a lancet tip to have a tapered surface with a point and a base. The tapered surface includes a plurality of different lancet gauge numbers corresponding to a plurality of different penetration depth increments. The method also includes securing the lancet tip to a support body.

In another aspect, the invention relates to a lancet including a lancet body and a sharp lancet tip extending from said lancet body. The sharp lancet tip preferably has a conical geometry having circular base at its point of connection with the lancet body, a point defining an included angle at a distal end thereof, and a tip height defined between the base and the point.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1A is a perspective view of a commercially-available lancet according to known form.

FIG. 1B is a side view of the lancet depicted in FIG. 1A.

FIG. 2A is a perspective view of a lancet according to an example embodiment of the present invention.

FIG. 2B is a side view of the lancet depicted in FIG. 2A.

FIG. 3 is a perspective view of an example lancet tip geometry of the lancet depicted in FIG. 2A.

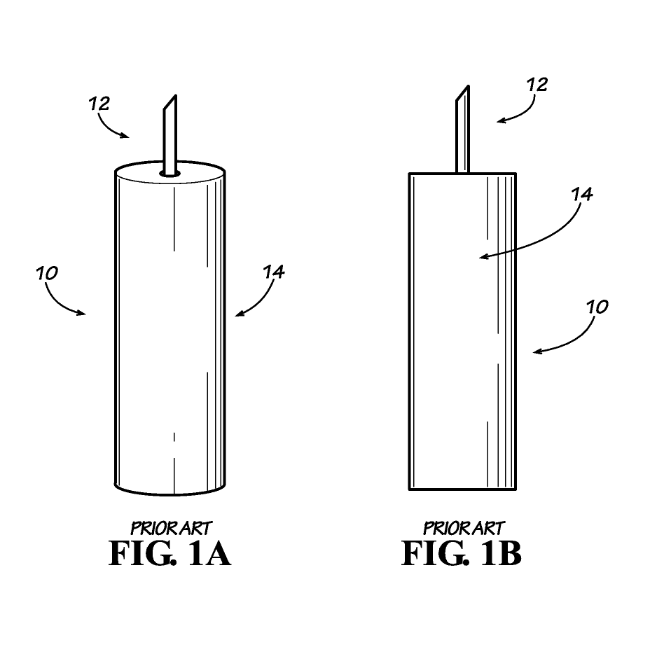

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

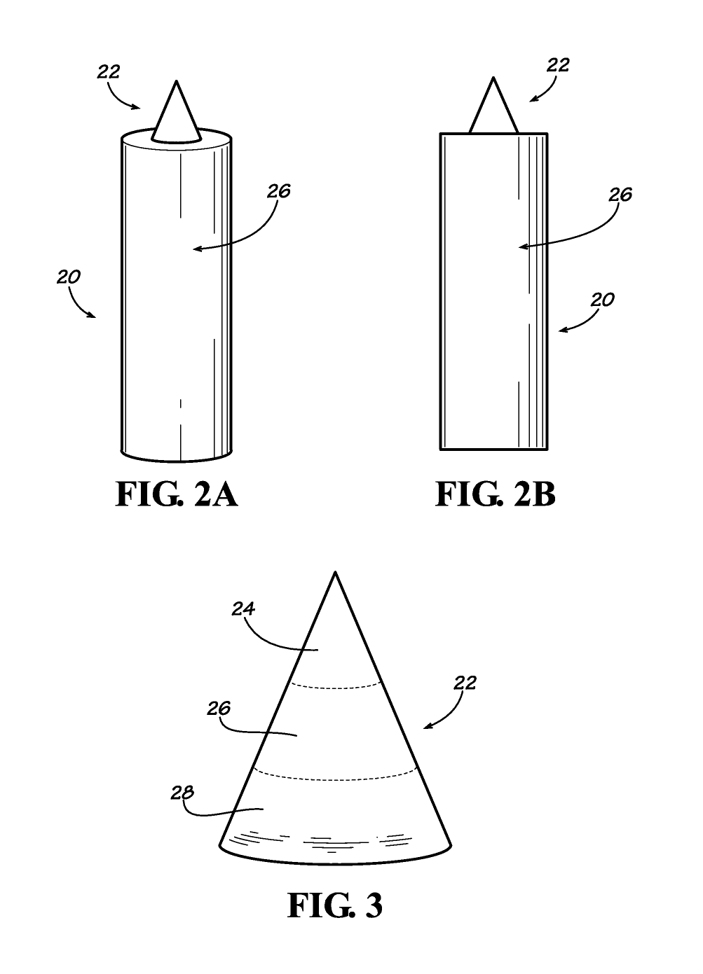

With reference now to the drawing figures, wherein like reference numbers represent corresponding parts throughout the several views, FIGS. 2A-3 show an improved lancet 20 according to an example embodiment of the present invention. The lancet of the present invention can be adapted for use within a single-use lancing device, for replaceable use in connection with a single-lancet multi-use lancing device, and/or included within a cartridge or other type of multi-lancet lancet device. As depicted, the lancet 20 includes a sharp conical tip 22 projecting from a lancet body 26. The lancet tip 22 is preferably formed of a hard polymeric material such as for example, polycarbonate, acrylic, polyethylene, Lucite™ or other polymethylmethacrylate, fiberglass, polyester, polyetherimide, polysulfone, polyether ether ketone, or other polymeric material capable of being molded, machined or otherwise formed into a substantially rigid sharp point. In alternate embodiments, a conical or generally conical tip formed of steel, aluminum, stainless steel, titanium, ceramic or other non-polymeric metals, ceramics, composite or other material(s) may be provided. The body 26, as depicted, has a generally cylindrical shape, but in alternate embodiments can comprise a rectangular elongate prism or other regular or irregular configurations. The body 26 is preferably formed of a polymeric material, and can be integrally formed with the lancet tip, for example by co-molding as a one-piece unitary component; or alternatively the lancet tip and body may be separately formed and attached to one another as by over-molding, press-fit, adhesive, thermal welding, snap connection(s) or other coupling(s) or attachment means.

As depicted, the sharp tip 22 has an inverted circular-circumference conical shape with a wide base and a narrow pointed tip, defining a tip height between the base and the tip of, for example, between about 2 mm-6 mm, or more preferably between about 3 mm-5 mm, or between about 3 mm-4 mm. Alternative shapes can include an inverted cone or pyramid with three sides forming a triangular base or an inverted cone or pyramid with four sides forming a square or rectangular base, or a generally conical element defining a sharp tip and concave or convex sides. Additional alternative shapes can include five or more sides forming a prismatic or faceted point or edge.

The tip 22 preferably has a point or edge that is sharp enough to prick the skin of a human or animal subject with minimal trauma and perception of pain, to generate a blood sample. The apex, edge or point of the sharp tip 22 preferably defines an acute angle of, for example, between about 2°-10°, or more preferably between about 3°-7°. This acute angle can continue to define the circumferential surface of the tip 22 from the point to the base, so that the tip is progressively tapered or stepped to vary and/or regulate the penetration depth of the tip into flesh, and thereby the size of the skin prick generated. Alternatively, the angle between diametrically-opposing) (180°) locations on the circumferential surface of the tip can vary between the point and the base. For example, at a certain vertical position between the point and the base, the angle can be smaller or larger than the acute angle of the point. In this manner, a single lancet format can be used universally in place of any of a range of standard lancet gauge formats, for differing sampling needs and/or for use at different sample sites. As particularly shown in FIG. 3, example widths are identified at points along the tip 22, which may generally correspond to smaller to larger gauges of standard lancet formats. For example, the slope or taper of the tip 22 is optionally designed so that the change in penetration depth into flesh is substantially equivalent to the change from one standard gauge to another standard gauge, in one or more increments, or along a smooth and continuously varying range. For example the narrowest region 24 can correspond to a larger gauge number, for example 33 gauge. The medium thickness region 26 can represent a medium gauge number, for example 28 gauge. The region of maximum thickness 28 can represent a small gauge number, for example 25 gauge. The design of the wall slope or taper allows a single lancet format to achieve necessary blood flow for different subjects or for different lancing regimens, simply by control of the penetration depth. This may eliminate the need for producing and stocking multiple different lancet formats for different lancing requirements. The size and geometry of the lancet tip 22 can vary depending on the particular application.

The lancet of the present invention may be particularly suited for adaptation and use with adjustable depth lancing devices of various forms. For example, in a method of use with a direct-link depth control lancing device, the penetration depth of the tip 22 of the lancet 20 is controlled, and the size of the skin prick is thereby varied depending on the desired sampling result. Incrementally increasing or decreasing the lancing depth correspondingly increases or decreases the dimension of the skin prick in the direction along the surface of the skin, thereby generating a larger or smaller sample size.

While the invention has been described with reference to preferred and example embodiments, it will be understood by those skilled in the art that a variety of modifications, additions and deletions are within the scope of the invention, as defined by the following claims.

What is claimed is:

1. A lancet comprising:
a lancet body; and
a lancet tip comprising generally conical tip element having a tapered surface extending between a sharp apex and a wider base, the tapered surface configured to define a plurality of different lancet gauge levels corresponding to a plurality of different penetration depths.

2. The lancet of claim 1, wherein the body tapered surface comprises a slope that is configured to define a change in penetration depth that is substantially equivalent to a change in gauge level.

3. The lancet of claim 1, wherein the body tapered surface comprises a consistent slope.

4. The lancet of claim 1, wherein the body tapered surface comprises a continuously-variable slope.

5. The lancet of claim 1, wherein the body comprises a conical geometry with a tip angle of between about 2°-10°.

6. The lancet of claim 1, comprising a tip height of between about 2 mm to about 6 mm.

7. A lancet comprising:
a lancet body; and
a sharp tip projecting from the lancet body and comprising a polymeric material, the sharp tip comprising an acutely-angled tapering outer surface and a base comprising a surface area that is larger than the sharp tip.

8. The lancet of claim 7, wherein the sharp tip is integrally formed with the lancet body as a unitary piece.

9. The lancet of claim 7, wherein the sharp tip outer surface has a smooth and continuous taper.

10. The lancet of claim 7, wherein the sharp tip has a substantially conical geometry.

11. The lancet of claim 1, wherein the sharp tip outer surface taper comprises a plurality of different penetration depth zones and a slope that is configured to change one standard lancet gauge level with a change in one penetration depth zone.

12. A method of controlling penetration depth of a lancet, the method comprising:
configuring a lancet tip to have a tapered surface with a point and a base, the tapered surface comprises a plurality of different lancet gauge numbers corresponding to a plurality of different penetration depth increments; and
securing the lancet tip to a support body.

13. The method of claim 12, wherein the lancet tip tapered surface is configured to change one lancet gauge number with a change of one penetration depth increment.

14. The method of claim 12, wherein the lancet tip tapered surface is conical.

15. The method of claim 13, wherein the lancet tip tapered surface comprises a circular base circumference.

16. A lancet comprising a lancet body and a sharp lancet tip extending from said lancet body, the sharp lancet tip comprising a conical geometry having circular base at its point of connection with the lancet body, a point defining an included angle at a distal end thereof, and a tip height defined between the base and the point.

17. The lancet of claim 16, wherein the sharp lancet tip comprises a polymeric material.
